(19) United States
(12) Patent Application Publication
(10) Pub. No.: US 2020/0114173 A1
(43) Pub. Date: Apr. 16, 2020

(54) RADIOTHERAPY SYSTEM AND METHOD USING THE SAME

(71) Applicant: Medical Intelligence Medizintechnik GmbH, Schwabmunchen (DE)

(72) Inventors: Andy LITTLE, West Sussex (GB); Antonio COSSU, Como (IT); Florian WEBER, Freising (DE); Per BERGFJORD, West Sussex (GB); Rui LIU, Augsburg (DE); Ulrike LUTZ, Landsberg (DE); Colin WINFIELD, West Sussex (GB)

(21) Appl. No.: 16/653,096

(22) Filed: Oct. 15, 2019

(30) Foreign Application Priority Data
Oct. 15, 2018 (EP) 18 200 423.4

(51) Int. Cl. A61N 5/10; A61B 6/04; A61B 6/00; A61B 90/00

(52) U.S. Cl. CPC A61N 5/1075; A61N 5/1081; A61N 5/1049; A61B 6/0407; A61B 6/54; A61B 2090/3937; A61B 90/39; A61N 2005/1076; A61N 2005/1074; A61N 2005/1059; A61N 2005/105; A61B 6/584

(57) ABSTRACT

According to an exemplary embodiment of the present disclosure, a determination of a (floating) isocenter of a radiotherapy system can be provided. For example, the radiotherapy system can comprises a patient support structure, a gantry configured to be rotatable around a gantry axis and having a radiation source, and at least one radiation imaging device. The system can include a calibration system comprising at least one first optical detector mounted at the gantry, at least one second optical detector fixed in a surrounding area of the patient support structure and/or the gantry, first fiducial markers selectively attachable at the patient support structure at defined positions and detectable by the first optical detector, and a phantom selectively attachable at the patient support structure at a defined position. The phantom can include second fiducial markers detectable by the second optical detector, and third fiducial markers configured to be detectable by the radiation imaging device. The system can comprises a controller configured to selectively activate the radiation source and rotate the gantry, and, for one or more rotational positions of the gantry, to determine a point of intersection of a beam axis of the radiation source and the gantry axis by linking detection data of the first optical detector, the second optical detector and/or the radiation imaging device.

### CROSS-REFERENCE TO RELATED APPLICATION(S)

This application relates to and claims priority from European Patent Application No. 18 200 423.4, filed on Oct. 15, 2018, the entire disclosure of which is incorporated herein by reference.

### FIELD OF THE DISCLOSURE

The present disclosure relates generally to real-time image guided radiation therapy. In particular, the exemplary embodiments of the present disclosure relate to a radiotherapy system and a method for operating a radiotherapy system.

### BACKGROUND INFORMATION

Medical imaging is commonly used to assist in the diagnosis and/or treatment of patients. X-Ray imaging is an example of a medical imaging technology that is often performed during the diagnosis and/or treatment of tumors. The treatment of tumors may be performed by using ionizing radiation provided by a linear accelerator (LINAC) generating a radiation beam of electrons and/or protons having particular energies. In such a radiotherapy system, the radiation beam should be aimed as precisely as possible at the target volume or target tissue, namely the tumor, while the adjacent healthy tissue should as far as possible not be irradiated.

Commonly, the radiation beam may be delivered by a radiation source configured to be moved in a circular orbit about the target volume to be irradiated. The radiation source may be moved to different positions in the circular orbit to deliver the radiation beam from these positions to the target volume. In this regard it is preferred that the radiation beam delivered from the different positions intersect in one single point, which is a reference point and which is commonly referred to as isocenter. In other words, the isocenter may be regarded as the point in space where radiation beams intersect when the radiation source is rotated during beam generation. In a radiotherapy system, it may therefore be desirable to verify the isocenter as accurately as possible before starting the treatment.

However, even with a previous verification of the isocenter, during the operation of the radiotherapy system, the accuracy of verifying the isocenter may be influenced by several factors. For example, the mass of the radiation source to be accelerated, the gravity acting on mechanical parts of the radiotherapy system etc. may be such factors. Accordingly, when moving the radiation source as explained above, deformation of at least the radiation source may occur. Although this deformation should be minor, it may be displace the isocenter and negatively affect the treatment outcome. However, even without such deformation, the isocenter may differ for different photon energies. With a displacing isocenter, a real path of the radiation beam delivered from the radiation source may deviate from its ideal path so that the target volume may not be hit as precisely as it is intended. As a result, radiation may also be delivered to healthy tissue.

Besides of a commonly known, so-called Winston-Lutz test, several approaches for verifying the isocenter are known in the art. For example, U.S. Pat. No. 8,488,862 describes ways to obtain a projection image of a phantom having a plurality of fiducials at known reference positions in a coordinate system associated with the phantom irradiated by a radiotherapy radiation source at a plurality of discrete locations of a trajectory path model. Then, a projection matrix from the projection image corresponding to each discrete location of the trajectory and the actual coordinate of the radiotherapy radiation source in the coordinate system associated with the phantom at each of the discrete locations based on the determined projection matrices are determined. Then, the trajectory path model of the radiotherapy radiation source to the determined actual position of the radiotherapy radiation source at the plurality of discrete locations is correlated.

However, there may be a need to provide a radiotherapy system and/or an operation method for a radiotherapy system that provides an improved verification of the isocenter, which address and/or overcome at least some of the deficiencies described herein above.

### SUMMARY OF EXEMPLARY EMBODIMENTS

According to an exemplary embodiment of the present disclosure, a radiotherapy system can be provided which is configured for diagnosing and/or treating a patient. The exemplary radiotherapy system may comprise e.g., a linear accelerator (LINAC) system together with an X-Ray imaging system and/or a magnetic resonance (MR) system and/or computed tomography (CT) system.

The exemplary radiotherapy system can comprises a patient support structure (e.g., a couch, etc.) which supports the patient to be diagnosed and/or treated. The system can further comprise a gantry which is configured to be rotatable around a gantry axis and having and/or carrying a radiation source. In particular, the gantry may be moved to different positions in the circular orbit to deliver a radiation beam from these positions to a target volume of the patient. The radiation source may comprise and/or may be referred to as a radiation head, a collimator, such as a multi-leaf-collimator (MLC), etc. In this exemplary context, the gantry itself may also be considered as the radiation source. The exemplary system can further comprise at least one radiation imaging device. The radiation imaging device may be provided as a kV and/or MV imaging device configured to provide 2D, 3D and/or 4D imaging.

The exemplary system can further comprise a determination and/or calibration system comprising at least one first optical detector mounted at the gantry. For example, the first optical detector may be or include a camera or the like. Further, the calibration system can comprise at least one second optical detector fixed in a surrounding area of the patient support structure and/or the gantry. For example, the second optical detector may be arranged on a wall or a ceiling of treatment room accommodating the radiotherapy system. The calibration system can further comprise a set of first fiducial markers which are selectively attachable at the patient support structure at defined positions and configured to be detectable by the first optical detector. Further, the calibration system can comprise a phantom which is selectively attachable at the patient support structure at a defined position, whereas the phantom can comprise a set of second fiducial markers configured to be detectable by the second optical detector and a set of third fiducial markers configured to be detectable by the radiation imaging device. The third fiducials markers may be arranged on defined positions at the phantom and may be visible for the radiation imaging device, such as an MV and/or a kV imager receiver or the like.

The exemplary radiotherapy system can further comprise a controller. The controller can be configured to at least selectively activate the radiation source and rotate the gantry, and, for one or more rotational positions of the gantry, to determine a point of intersection of a beam axis of the radiation source and the gantry axis by linking detection data of at least the first optical detector, the second optical detector and/or the radiation imaging device. The point of intersection may be referred to as a (floating) isocenter. The exemplary controller may comprise a processor, a memory etc. for processing and/or storing e.g., imaging data, calibration data, such as isocenter determination data, or the like. The controller may be connected to other parts of the systems by suitable data communication lines.

Several advantages may be achieved with this exemplary configuration of the radiotherapy system according to the exemplary embodiment of the present disclosure. For example, the (floating) isocenter may be precisely and accurately determined for each single rotational angle, i.e. the current rotational position relative to the target, although the isocenter may be different for different rotational angles due to mechanical deformation caused by the weight of the gantry or the gravitational force. Further, the system may improve precise verification of the (floating) isocenter, while reducing exposure of the patient to X-rays. In particular, a dose calibration, X-Ray imaging calibration and verifying the floating isocenter with gantry rotation calibration may be performed using the phantom and a one-time gantry rotation, e.g., by rotating the gantry 360° about the gantry axis. In a common system, however, if the target is outside of the tube, patient re-positioning with a movement of the patient support structure would be needed, and afterwards an imaging by use of a radiation imaging device and re-mapping with CT images would start again. Consequently, in a common system, the patient would be exposed to a high level of radiation exposure just in order to be moved into a proper position. In contrast thereto, in the system according to this invention, the optical detector may allow a table movement without using e.g., X-Rays by using e.g., IR-markers to guide the patient support structure movement, and an extended field of view. Therefore, various exemplary embodiments of the present disclosure may provide a non-invasive position and/or isocenter verification, and reduces set-up time of the patient.

According to another exemplary embodiment of the present disclosure, the controller can be further configured to control a position and/or orientation of the patient support structure to align it at the determined point of intersection, namely the currently determined (floating) isocenter. The patient support structure may have six degrees of freedom for its own movement and may further be driven by a controllable drive. Thus, if it is determined that the radiation beam is not (or no more) aligned with the target, this undesirable deviation can be compensated by controllable moving the anyway movable patient support structure. In other words, the system may be configured to follow the current (floating) isocenter.

According to a further exemplary embodiment of the present disclosure, the controller may further be configured to, based on an image of the phantom captured by the at least one radiation imaging device, calibrate a beam shaper of the radiation source at each rotational position of the gantry. The beam shaper may be a multi-leaf-collimator (MLC) or the like. Thus, the beam shaper may be adjusted to the respective currently determined (floating) isocenter, especially in real-time, so that even slight deformations of the gantry or other mechanical parts of the radiotherapy system and a resulting floating isocenter can be taken into account.

In another exemplary embodiment of the present disclosure, the controller may be further configured to, based on an image of the phantom captured by the at least one radiation imaging device, determine in real-time a radiation dose to be delivered by the radiation source at each rotational position of the gantry. Thus, e.g., an on-line dose adaption dependent on the current isocenter may be performed.

In yet another exemplary embodiment of the present disclosure, the controller may be further configured to, based on an image of the phantom captured by the at least one radiation imaging device, determine an offset of the gantry at each rotational position of the gantry. Thus, the (floating) isocenter may be determined even more precisely at each rotational angle of the gantry.

According to still another exemplary embodiment of the present disclosure, the third fiducial markers may be embedded in a material of the phantom to be visible by use of the at least one radiation imaging device. Thus, since fiducial markers are embedded in the phantom and be visible for MV and/or kV imager receivers, projected dots at 2D plane can be used for calculating the gantry offset at each rotation angle.

According to a further exemplary embodiment of the present disclosure, the phantom may be linked to the at least one first optical detector. It may be linked via suitable data communication lines, the controller and/or a software-based control. Thus, determining the current (floating) isocenter may be performed even more precisely.

In still a further exemplary embodiment of the present disclosure, the phantom, via the second optical detector, may be linked to at last one absolute position in a free three-dimensional space. Thus, determining the current (floating) isocenter may be performed even more precisely. The phantom is not only used for geometry calibration but may be also used for beam and imaging calibration.

According to another exemplary embodiment of the present disclosure, the phantom may further comprise a further set of first fiducial markers configured to be detectable by the at least one first optical detector. Thus, the phantom may be linked, via the first fiducial markers, to the first optical detector.

In yet another exemplary embodiment of the present disclosure, a further first optical detector, or a similar means, may be mounted at the at least one radiation imaging device to be arranged opposite to the first optical detector mounted at the gantry. Thus, corresponding first fiducial markers may be detectable even if the gantry is currently located on a side facing away from the top of the patient support structure.

According to a still further exemplary embodiment of the present disclosure, further first fiducial markers may be selectively attachable at a bottom side of the patient support structure. Thus, the first optical detector may detect one of the first fiducial markers even if the gantry is currently located on a side facing away from the top side or, respectively, facing the bottom side of the patient support structure.

In an additional exemplary embodiment of the present disclosure, first fiducial markers can be carried by at least one frame structure which can be mounted along a top side of the patient support structure at different attachment positions. Thus, even if the phantom has already been removed before loading the patient on the top side of the patient support structure, the target tumor position can be well determined based on the kV imager and previous imaging calibration, since the reference frame structure is still detectable and/or trackable by the first optical detector mounted at least at the gantry.

The first optical detector may be an IR-camera. Thus, a cost-effective and small-sized possibility for the marker detection is possible. The second optical detector may be a laser device. For example, the second optical detector may be a laser tracker or the like. Thus, the phantom is linkable with pre-defined absolute position(s) at 3D free space.

In another exemplary embodiment of the present disclosure, a further set of the first fiducial markers may be provided via a support structure attachable to a patient and wherein these first fiducial markers are linked to the first fiducial markers attached to the patient support structure. Thus, co-registering with the other first fiducial markers, e.g., mounted at the reference frame structure, is possible which may allow real-time motion detection of the patient. Ideally, this information may be provided to the controller to control calibration and/or treatment accordingly.

According to a further exemplary embodiment of the present disclosure, the controller may further be configured, for the one or more rotational positions of the gantry, to determine a current or predicted position of the patient support structure and, via the first optical detector and the first fiducial markers, to determine a current or predicted position of the gantry relative to the determined current or predicted position of the patient support structure.

Thus, a collision between the gantry and the patient support structure can be avoided. In particular, since the first optical detector is mounted at the gantry, a distance between e.g., the top side of the patient support structure and the gantry may be determined, at each gantry angle during calibration. At each gantry angle, even all possible locations and/or positions of the patient support structure may be known, e.g., from a simulation from a 3D CAD model or the like. Since the first fiducial markers are arranged at a pre-defined position on the top side of the patient support structure, a minimum safety distance between the first optical detector and the first fiducial markers may be calculated and/or simulated. After calibration, a table-look-up approach can be applied for each optical detector.

In another exemplary embodiment of the present disclosure, the controller may be further configured to, based on determining a mechanical center of the gantry via the first optical detector, determine a current or predicted angular velocity and/or acceleration. Thus, the first optical detector mounted at the gantry may be used to determine the angular velocity and/or acceleration of the rotating gantry. For example, after calibration, the gantry mechanical center is known by use of the first optical detector, for each gantry and/or position of the patient support structure. Since the weight or mass of the gantry, e.g., its head and arm, is known, e.g., from a 3D CAD model or the like, it may be determined what level of energy is needed to drive or stop the gantry to a target angle with a desired speed and acceleration. For example, the gantry can be currently at a 6 o'clock position and it desired to stop it at a 12:00 o'clock position, gravity, inertia and the like may be used to set optimized settings for acceleration, drive and/or braking.

Further, according to yet another exemplary embodiment of the present disclosure, a method for operating a radiotherapy system can be provided, which comprises the following steps:
providing a set of first fiducial markers at the patient support structure (e.g., the couch) of the radiotherapy system at defined positions,
providing the phantom at the patient support structure at a defined position, wherein the phantom comprises the set of second fiducial markers and the set of third fiducial markers,
detecting the first fiducial markers by the first optical detector mounted on the gantry which is configured to be rotatable around a gantry axis and having a radiation source,
detecting the second fiducial markers by the second optical detector fixed in the surrounding area of the patient support structure and/or the gantry,
detecting the third fiducial markers by the radiation imaging device of the radiotherapy system,
controlling the radiation source to be at least selectively activated and controlling the gantry to be rotated, and
for one or more rotational positions of the gantry, determining a point of intersection of a beam axis of the radiation source and the gantry axis by linking detection data of at least the first optical detector, the second optical detector and/or the radiation imaging device.

For example, the first fiducial markers and the phantom may be provided at e.g., the patient support structure top to have a reproducible position. The second optical detector may be arranged on e.g., a wall or ceiling of a room accommodating the radiotherapy system, so as to link with pre-defined absolute positions in 3D free space. Then, the radiation source is activated to generate a radiation beam while the gantry is controlled to be rotated, e.g., about 360° about the gantry axis, so as to link the phantom to the first optical detector and/or the radiation imaging device, such as a MV image receiver panel, at each gantry angle position. It is noted that, since the third fiducial markers are arranged precisely at the phantom and visible for e.g., MV and/or kV imager receivers, projected dots at 2D plane may be used for calculating a gantry offset at each rotation angle. Therefore, the (floating) isocenter may be determined at each gantry angle. Similarly, such information can also be linked to the first optical detector during calibration, because the introduced first fiducial markers are adapted to provide a large field of view (FoV) as compared to e.g., a common Winston-Lutz ball approach.

In a further exemplary embodiment of the present disclosure, the phantom image may be used to calibrate a beam shaper of the radiation source, such as a MLC, and to determine a proper X-Ray imaging at each angle. For example, the phantom can be removed from the couch and the patient is loaded. Thus, since the first fiducial markers may still be tracked with the one or more first optical detector mounted on the gantry, the target tumor position may be precisely determined based on the kV imager and previous imaging calibration. By using more than one first fiducial markers a single-fault problem may be avoided, so that the above system may also be suitable for e.g., multi-fraction non-coplanar LINAC treatment. In addition, further first fiducial markers may be arranged at the patient body to co-register with the first fiducial markers provided at the couch such that real-time motion of the patient may be monitored.

According to another exemplary embodiment of the present disclosure, the patient support structure may be driven to follow the determined (floating) isocenter at each gantry angle. Further, e.g., the radiation source may be controlled in real-time to adjust the beam shaping on basis of the determined (floating) isocenter. In addition or alternatively, an on-line dose adaption of the radiation dose may be performed.

It should be noted that embodiments as described above may be combined with respect to each other so as to gain a synergetic effect, which may extend over the separate technical effects of the single features. Likewise, the above method can be modified by the embodiments of the above radiotherapy system and vice versa.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a perspective view of a radiotherapy system according to an exemplary embodiment of the present disclosure;

FIG. 2 is a side view of an exemplary phantom according to an exemplary embodiment of the present disclosure;

FIG. 3 is a perspective view of the exemplary radiotherapy system of FIG. 1, in which a phantom has been removed and replaced by a patient to be diagnosed and/or treated;

FIG. 4 is a schematic front view of the exemplary radiotherapy system shown in FIG. 1; and

FIG. 5 is a flow diagram of a method of operating the radiotherapy system shown in FIG. 1, according to an exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

In the following, a detailed description of exemplary embodiments of the present disclosure will be described in further detail.

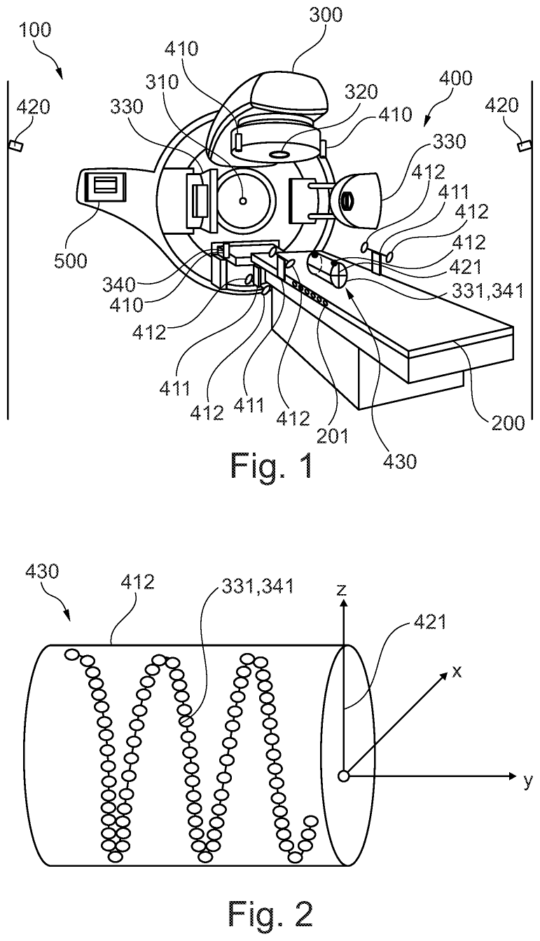

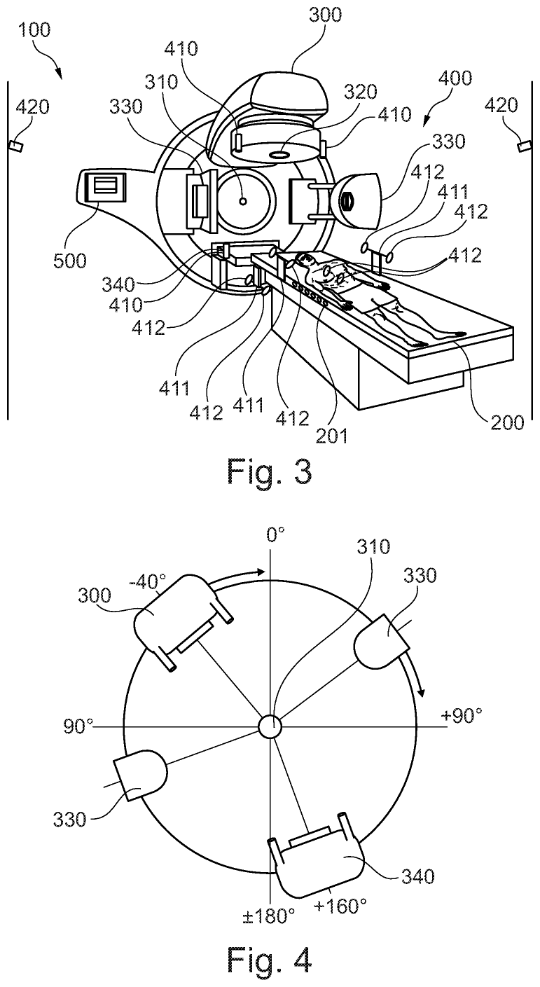

FIG. 1 illustrates schematically a radiotherapy system 100, which, in some exemplary embodiments, may be configured as a linear accelerator (LINAC) radiotherapy system. The exemplary system 100 can comprise a patient support structure which can be or include, e.g., a couch 200 configured to support a recumbent patient on a patient support structure/couch top (see FIG. 3). The couch is electronically controllable and movable with six degrees of freedom. The exemplary system 100 can further comprise a gantry 300 having a gantry arm 311 and a gantry head 312. The gantry 300 can be rotatable around a gantry axis 310 and has a radiation source 320. The radiation source 320 may comprises at least one beam shaper, such as a multi leaf collimator (MLC), and may provide or produce X-rays and/or high energy electrons or photons in form of a radiation beam for medicinal purposes in radiation therapy. The system 100 can further comprise at least one radiation imaging device 330, 340, wherein, in some exemplary embodiments, a first radiation imaging device 330 may be configured as a kV imager and a second radiation imaging device 340 may be configured as a MV imager. In the exemplary embodiment shown in FIG. 1, the first radiation imaging device 330 comprises an imager radiation source and an imager receiver panel. Likewise, but offset from the first radiation imaging device 330, the second radiation imaging device 340 comprises an imager radiation source and an imager receiver panel.

As illustrated in FIG. 1, the system 100 can further comprise a determination or calibration system 400 configured to at least determine an isocenter, which may be regarded as a point of intersection of a radiation beam axis of the radiation source 320 and the gantry axis 310. The determined (floating) isocenter may be the location in which the target volume to be irradiated is to be arranged. At the gantry 300, at least one first optical detector 410 is mounted, wherein, in this embodiment, two exemplary first optical detector 410 in form of infrared/IR cameras are provided. The first optical detector 410 are fixedly mounted near the radiation beam outlet of the radiation source 320 so that they may participate any movement, e.g., the rotation of the gantry 300, but also any deformation of the gantry 300, e.g., of the gantry arm 311 and/or gantry head 312, due to its weight, gravity, centrifugal force, etc. In some exemplary embodiments, at least one further first optical detector 410 may be arranged at a side of the system 100 opposing the first optical detector 410 mounted at the gantry 300. As illustrated in FIG. 1, the further first optical detector 410 are mounted at the receiver panel of the second radiation imaging device 340.

In a surrounding area of the couch 200 and/or the gantry 300, at least one second optical detector 420 of the system 100, in particular the system 400 is fixed in place. The surrounding area may be a wall, as shown in the exemplary embodiment of FIG. 1, a ceiling etc., of a room accommodating the system 100. By way of example, only two second optical detector 420 are shown, however, one, three, four, five, six or more can be provided and aligned to the system 100, in particular the couch 200 or the couch top. The second optical detector may be a laser tracker.

Further, the system 100, and, e.g., the system 400 can comprise a set of first fiducial markers 412 which are selectively attachable and detachable at the couch 200 at defined positions and configured to be detectable by the first optical detector 410. In certain exemplary embodiments, the patient support structure (e.g., the couch 200) can comprise several attachment positions 201, e.g., engaging holes, so that the first fiducial markers 412 may be mounted along a top side of the couch 200 at different positions. In some exemplary embodiments, the first fiducial markers 412 are attached to e.g., a reference frame structure 411 which is mountable at an edge region of the couch 200 and extends away from the couch top. In further exemplary embodiments, on a bottom side of the couch 200, a further set of first fiducial markers 412 may be attached to a further reference frame structure 411.

The system 100, e.g., the system 400 can further comprise a phantom 430 which is selectively attachable to and detachable from the couch 200 at a defined position. The couch top may comprise reference points, lines etc. to facilitate alignment of the phantom, as indicated in FIG. 1 by a dashed line. As shown in FIG. 1, the phantom 430 can be or include a tube and comprises a set of second fiducial markers 421 configured to be detectable by the second optical detector 420, e.g., the laser tracker. In some exemplary embodiments, the phantom 430 may be linked to at last one absolute position in free three-dimensional space via the second optical detector 420 is linked. The phantom 430 can further comprise a set of third fiducial markers 331, 341 configured to be detectable by the radiation imaging device 330 and/or 340. In some exemplary embodiments, the third fiducial markers 331, 341 may be embedded in a material of the phantom 430 to be visible by use of the at least one of the radiation imaging devices 330, 340. In some exemplary embodiments, the phantom 430 may further comprise a further set of the first fiducial markers 412 so that the phantom 430 may be linked to the at least one first optical detector 410.

The system 100 further comprises a controller 500 which may be an electronic device comprising a processor, a physical memory etc. In particular for calibrating the system 100 and/or determining a (floating) isocenter of the system 100, the controller 500 can be configured to selectively activate the radiation source 320 and rotate the gantry 300 about the gantry axis 310, around any angle section or e.g., 360°. This rotation may be also be referred to as a movement in a circular orbit about a target volume to be irradiated. Further, the controller 500 is configured to, for one or more rotational positions of the gantry 300 relative to the starting point of rotation, determine a point of intersection, e.g., the (floating) isocenter, of the beam axis of the radiation source 320 and the gantry axis 310 by linking detection data of at least the first optical detector 410, the second optical detector 420 and/or the radiation imaging device 330, 340.

In some exemplary embodiments, the controller 500 may be further configured to control a position and/or orientation of the couch 200 to align it at the determined point of intersection, e.g., the (floating) isocenter. The controller 500 may further configured to, based on an image of the phantom 430 captured by the at least one radiation imaging device 330, 340, calibrate the beam shaper of the radiation source 320 at each rotational position of the gantry 300. In addition or alternatively, the controller 500 may be further configured to, based on an image of the phantom 430 captured by the at least one radiation imaging device 330, 340, determine a radiation dose to be delivered by the radiation source 320 at each rotational position of the gantry 300. Further, in some exemplary embodiments, the controller 500 may further be configured to, based on an image of the phantom 430 captured by the at least one radiation imaging device 330, 340, determine an offset of the gantry 300 at each rotational position of the gantry 300. In some exemplary embodiments, the controller 500 may further be configured, for the one or more rotational positions of the gantry 300, to determine a current or predicted position of the couch 200. If the position of the couch 200 is known, via the first optical detector 410 and the first fiducial markers 412, the controller 500 may determine a current or predicted position of the gantry 300 relative to the determined current or predicted position of the couch 200. Further, in some exemplary embodiments, the controller 500 may be further configured to, based on determining a mechanical center of the gantry 300 via the first optical detector 410, determine a current or predicted angular velocity and/or acceleration.

FIG. 2 shows the exemplary phantom 430 as a single part which is exemplarily formed as the tube and comprises the set of second fiducial markers 421 configured to be detectable by the second optical detector 420, e.g., the laser tracker, the set of third fiducial markers 331, 341 embedded in a material of the phantom 430 to be visible by use of the at least one of the radiation imaging devices 330, 340 and configured to be detectable by the radiation imaging device 330 and/or 340. In this exemplary embodiment, the phantom 430 can further comprise the further set of the first fiducial markers 412 so that the phantom 430 may be linked to the at least one first optical detector 410.

As illustrated in FIG. 3, the exemplary phantom 430 has been removed from the top side of the couch 200. Instead, as schematically indicated, a patient has been placed on the top side of the couch 200. In some exemplary embodiments, the patient may wear a support structure 413, e.g., a textile part or the like, where a further set of the first fiducial markers 412 is attached. This further set of the first fiducial markers 412 is linked to the other first fiducial markers 412 attached to the couch 200. In some exemplary embodiments, the controller 500 may be configured to detect motion of the patient via the first optical detecting means 410 by detecting e.g., deviations in positional deviations between the patient-sided first fiducial markers 412 and the couch-sided first fiducial markers 412.

FIG. 4 shows a schematic front view of the system 100, in which the couch 200 is hidden for better illustration. As discussed herein, the controller 500 may be configured to control the angular velocity and/or acceleration of the gantry 300. For that, the first optical detector 410 mounted at the gantry 300 may be used to determine the angular velocity and/or acceleration of the rotating gantry 300. After calibration of the system 100, the gantry 300 mechanical center is already known by use of the first optical detector 410, for each gantry and/or couch position. Since the weight or mass of the gantry 300, e.g., its gantry head and arm, is known, e.g., from a 3D CAD model, trigonometry, or the like, it may be determined what level of energy is needed to drive or stop the gantry 300 to a target angle with a desired speed and acceleration. In the example according to FIG. 4, the gantry 300 is to be moved from its starting point at about −40° to its end point at about +160°.

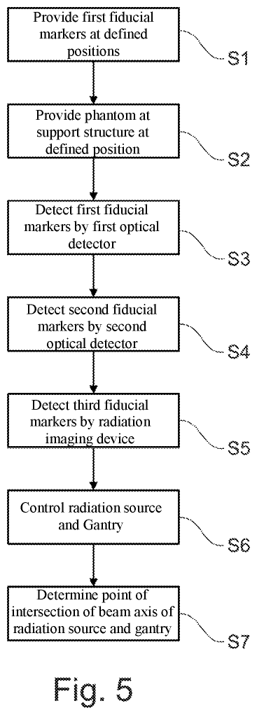

With reference to the flow chart shown in FIG. 5, an operation and/or a calibration of the radiotherapy system 100 may be as described below.

For example, in step S1, the set of first fiducial markers 412 can be provided, e.g., mounted, at the couch 200 at the defined positions. These positions may vary in dependency from the patient, the target volume, the location of the target volume etc. In step S2, the phantom 430 is provided, e.g., mounted and/or aligned with a reference mark or the like, at the couch 200 at a defined position, whereas the phantom 430 comprises the set of second fiducial markers 421 and the set of third fiducial markers 331, 341. In step S3, the first fiducial markers 412 can be detected by the first optical detector 410 mounted on the gantry 300 which can be configured to be rotatable around the gantry axis 310 and carrying the radiation source 320. In step S4, the second fiducial markers 421 can be detected by the second optical detector 420 fixed in a surrounding area of the couch 200 and/or the gantry 300. In step S5, the third fiducial markers 331, 431 can be detected by a radiation imaging device 330, 340 of the radiotherapy system 100. In step S6, the radiation source 320 can be controlled to be selectively activated and the gantry 320 is controlled to be rotated. In step S7, for one or more rotational positions of the gantry 300, the point of intersection of the beam axis of the radiation source 320 and the gantry axis 310, e.g., the (floating) isocenter, can be determined by linking detection data of at least the first optical detector 410, the second optical detector 420 and/or the radiation imaging device 330, 340.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

What is claimed is:

1. A radiotherapy system, comprising
a patient support structure;
a gantry which is (i) rotatable around a gantry axis, and (ii) including a radiation source;
at least one radiation imaging device;
a calibration system comprising:
at least one first optical detector provided on the gantry,
at least one second optical detector provided in a surrounding area of at least one of the patient support structure or the gantry,
first fiducial markers (i) selectively attachable at the patient support structure at first predetermined positions, and (ii) configured to be detectable by the first optical detector, and
a phantom selectively attachable at the patient support structure at a second predetermined position, wherein the phantom comprises (i) second fiducial markers detectable by the second optical detector, and (ii) third fiducial markers detectable by the radiation imaging device; and
a controller which is configured to:
selectively activate the radiation source,
rotate the gantry, and
for one or more rotational positions of the gantry, determine a point of intersection of a beam axis of the radiation source and the gantry axis by linking detection data of at least one of the first optical detector, the second optical detector or the radiation imaging device.

2. The radiotherapy system of claim 1, wherein the controller is further configured to control at least one of a position or an orientation of the patient support structure to align the patient support structure at the determined point of intersection.

3. The radiotherapy system of claim 1, wherein the controller is further configured to, based on at least one image of the phantom obtained by the at least one radiation imaging device, calibrate a beam shaper of the radiation source at each rotational position of the gantry.

4. The radiotherapy system of claim 1, wherein the controller is further configured to, based on an image of the phantom obtained by the at least one radiation imaging device, calibrate the radiation imaging device at each rotational position of the gantry.

5. The radiotherapy system of claim 1, wherein the controller is further configured to, based on an image of the phantom obtained by the at least one radiation imaging device, determine an offset of the gantry at each rotational position of the gantry.

6. The radiotherapy system of claim 1, wherein the third fiducial markers are embedded in a material of the phantom to be visible by a usage of the at least one radiation imaging device.

7. The radiotherapy system of claim 1, wherein the phantom is linked to the at least one first optical detector.

8. The radiotherapy system of claim 1, wherein the phantom is associated with at last one absolute position in a free three-dimensional space via the second optical detector.

9. The radiotherapy system of claim 1, wherein the phantom further comprises a further set of the first fiducial markers which are configured to be detected by the at least one first optical detector.

10. The radiotherapy system of claim 1, wherein the first optical detector comprises a further optical detector which is mounted at the at least one radiation imaging device to be arranged opposite to the first optical detector mounted at the gantry.

11. The radiotherapy system of claim 1, wherein the first fiducial markers includes a further set of markers which are selectively attachable at a bottom side of the patient support structure.

12. The radiotherapy system of claim 1, wherein the first fiducial markers are supported by at least one frame structure which is mountable along a top side of the patient support structure at different attachment positions.

13. The radiotherapy system of claim 1, wherein the first optical detector is an infra-red (IR) camera.

14. The radiotherapy system of claim 1, wherein the second optical detector is a laser device.

15. The radiotherapy system of claim 1, wherein the first fiducial markers includes a further set of markers which is provided via a support structure that is attachable to a patient, and wherein the further set of markers are linked to the first fiducial markers which are attached to the patient support structure.

16. The radiotherapy system of claim 1, wherein the controller is further configured to:
for the one or more rotational positions of the gantry, to determine at least one of a current position or a predicted position of the patient support structure, and
via the first optical detector and the first fiducial markers, to determine at least one of a current position or a predicted position of the gantry relative to the determined current position or the determined predicted position of the patient support structure.

17. The radiotherapy system of claim 1, wherein the controller is further configured to, based on a determination of a mechanical center of the gantry via the first optical detector, determine at least one of (i) a current angular velocity or a predicted angular velocity or (ii) an acceleration of the gantry.

18. A method for operating a radiotherapy system, comprising:
providing first fiducial markers at a patient support structure of the radiotherapy system at first predetermined positions;
providing a phantom at the patient support structure at a second predetermined position, wherein the phantom comprises second fiducial markers and third fiducial markers;
detecting the first fiducial markers by a first optical detector provided on a gantry which is rotatable around a gantry axis and having a radiation source;
detecting the second fiducial markers by a second optical detector fixed in a surrounding area of at least one of the patient support structure or the gantry;
detecting the third fiducial markers by a radiation imaging device of the radiotherapy system;
controlling the radiation source to be selectively activated;
controlling the gantry to be rotated; and
for one or more rotational positions of the gantry, determining a point of intersection of a beam axis of the radiation source and the gantry axis by linking detection data of at least one of at least the first optical detector, the second optical detector or the radiation imaging device.
